(19) United States
(12) Patent Application Publication
(10) Pub. No.: US 2026/0277362 A1
(43) Pub. Date: Sep. 17, 2026

(54) TOUCH SENSOR APPARATUS FOR A MEDICAL DEVICE, MEDICAL DEVICE AND METHOD FOR OPERATOR CONTROL OF A TOUCH SENSOR APPARATUS FOR A MEDICAL DEVICE

(71) Applicant: Siemens Healthineers AG, Forchheim (DE)

(72) Inventor: Ulrich BATZER, Spardorf (DE)

(73) Assignee: Siemens Healthineers AG, Forchheim (DE)

(21) Appl. No.: 19/565,681

(22) Filed: Mar. 13, 2026

(30) Foreign Application Priority Data
Mar. 14, 2025 (DE) 10 2025 109 846.8

(51) Int. Cl. G06F 3/041; A61B 6/00; G01R 31/28; G06F 3/044; G06F 3/045

(52) U.S. Cl. CPC G06F 3/0418; A61B 6/548; G01R 31/2829; G06F 3/0414; G06F 3/04166; G06F 3/044; G06F 3/045; G06F 2203/04106

(57) ABSTRACT

One or more example embodiments relates to a touch sensor apparatus for a medical device, comprising a touch sensor, a controller configured to detect an operator control event at a user interface of the touch sensor apparatus by evaluating sensor data of the touch sensor, and a fault detector configured to detect a fault relating to the controller. The fault detector is configured to at least one of: (a) measure the common mode interference directly if the fault is caused by a common mode interference; or, generate an alternating current voltage signal if the fault is caused by a defect in the controller, apply said signal to the touch sensor, detect an influence caused by the operator control event, and check whether the operator control event is detectable via the fault detector and not via the controller.

### CROSS-REFERENCE TO RELATED APPLICATION(S)

The present application claims priority under 35 U.S.C. § 119 to German Patent Application No. 10 2025 109 846.8, filed Mar. 14, 2025, the entire contents of which is incorporated herein by reference.

### FIELD

One or more example embodiments relates to a touch sensor apparatus for a medical device, a medical device comprising a touch sensor apparatus of said type, and a method for operator control of a touch sensor apparatus of said type for a medical device of said type.

### RELATED ART

A medical device may have at least one safety-critical function for which at least one safety measure is provided. The safety-critical function may for example comprise an activation of an irradiation using X-ray radiation, gamma radiation or another type of radiation that is used for a medical application. The safety measure is intended for example to prevent an operator control event of an operator control apparatus that is associated with the medical device from being detected even though said operator control event has not been carried out by a user of the medical device or not carried out as detected. Alternatively or in addition thereto, the safety measure is intended for example to prevent the operator control event from not being detected due to an unnoticed defect of the operator control apparatus.

The safety measure is particularly relevant when the operator control apparatus is a touch sensor apparatus that for example comprises a capacitive or resistive touch sensor which is designed to detect an operator control event on a user interface of the touch sensor apparatus. The reason for this is for example that a detection principle of the touch sensor apparatus may be sensitive to interferences from an environment such that a control unit of the touch sensor apparatus may for example deliver incorrect results due to the interference. Alternatively or in addition thereto, the touch sensor apparatus may be more susceptible to electronic defects than for example a mechanical operator control apparatus such as a key or a button, such that a defect may occur in the control unit unexpectedly at any time.

### SUMMARY

One or more example embodiments provides a solution via which a fault relating to a control unit (also known as a controller) of a touch sensor apparatus can be reliably detected.

### BRIEF DESCRIPTION OF THE DRAWINGS

In the drawings:

FIG. 1 shows a schematic view of a medical device comprising a touch sensor apparatus according to one or more example embodiments,

FIG. 2 shows a schematic view of a feature (a) for a fault detection facility according to one or more example embodiments, and

FIG. 3 shows a schematic view of a feature (b) for a fault detection facility according to one or more example embodiments.

Functionally equivalent elements in the figures are labeled with the same reference signs.

### DETAILED DESCRIPTION

One or more example embodiments relates to a touch sensor apparatus for a medical device. The touch sensor apparatus is an apparatus that is designed at least to detect whether an object and/or a person has come into contact with a user interface of the touch sensor apparatus. The touch sensor apparatus may alternatively be referred to as a tactility sensor apparatus or tactile sensor apparatus. The touch sensor apparatus comprises at least one touch sensor, a control unit and a fault detection facility (also known as a fault detector) for detecting a fault relating to the control unit. The control unit is designed to detect an operator control event at or on the user interface of the touch sensor apparatus by evaluating sensor data of the touch sensor. The touch sensor may for example comprise a capacitive and/or a resistive sensor and/or a force sensor. The touch sensor therefore responds for example to touch, to the effect of force and/or to pressure, where touch, the effect of force and/or pressure are in each case understood as at least part of an operator control event. The touch sensor apparatus may therefore have recourse to different techniques known for touch sensor apparatuses for detecting the operator control event. The touch sensor apparatus may alternatively be referred to as a touch sensor or touch-sensitive sensor. The control unit may alternatively be referred to as a touch controller or touch control device. The sensor data detected by the touch sensor may be transmitted within the touch sensor apparatus to the control unit and be evaluated by the latter in order to establish and therefore detect the operator control event or in order to establish and therefore detect no operator control event.

According to a feature (a), in the event that the fault is caused by common mode interference, the fault detection facility is designed to measure the common mode interference directly. The common mode interference is to be understood as an interference voltage and/or as an interference current which for example is generated by another apparatus which is not part of the touch sensor apparatus and/or the medical device which comprises the touch sensor apparatus or with which the touch sensor apparatus is associated and for example can affect, in particular corrupt, the sensor data received by the control unit. The other apparatus, which is designed for example as a further medical device, generates for example voltage signals at frequencies which lie at least in part in a same frequency range, such as for example an electrical field, which in the case of the capacitive touch sensor is applied at the user interface in order to enable the operator control event to be detected. The common mode interference therefore affects the sensor data that the touch sensor transmits to the control unit. The common mode interference may alternatively be referred to as common-mode noise.

The fault detection facility is designed to measure the common mode interference directly by detecting a voltage which dips between a first reference potential referenced to a ground of the control unit and a second reference potential referenced to a ground of an insulated power supply unit of the touch sensor apparatus. The fault detection facility is therefore designed to measure a difference between the first reference potential and the second reference potential, the measured difference being the detected voltage. The insulated power supply unit is to be understood as a component of the touch sensor apparatus which, in relation to the rest of the touch sensor apparatus, in particular in relation to the control unit, is electrically insulated. The insulated power supply unit is therefore grounded for example such that the ground referenced to the insulated power supply unit can be for example a ground potential. The ground of the control unit is likewise to be understood as a kind of grounding. The ground of the control unit can for example likewise be grounded.

Feature (a) is based at least on the knowledge that a wanted signal that describes the operator control event at the user interface is transmitted only between the touch sensor and the control unit, but not further to a section of the touch sensor apparatus insulated for example from the touch sensor. The first reference potential and the second reference potential taken into account during the detection of the voltage are therefore each unaffected by the wanted signal but are affected by the common mode interference. This is because the common mode interference is transmitted from the control unit to further components and/or electrical connections within the touch sensor apparatus. As soon as the common mode interference occurs, it can therefore be detected via the fault detection facility unaffected by other signals such as the wanted signal since the fault detection facility is arranged between the control unit and the insulated power supply unit and is therefore located in the insulated section of the touch sensor apparatus. If the common mode interference is detected, it can be inferred that a fault due to the common mode interference is at least to be expected, in particular is present. Since the common mode interference affects the wanted signal, a fault relating to the control unit is then present since the detection accuracy of the operator control event is affected and consequently prone to error.

The insulated section may be understood as that area of the touch sensor apparatus that is electrically insulated, and therefore decoupled, at least from the control unit and the touch sensor as well as, where applicable, from further components of the touch sensor apparatus, such as, for example, an energy supply unit of the touch sensor apparatus.

According to a feature (b), the fault detection facility is alternatively or additionally designed to generate an alternating current voltage signal if the fault is caused by a defect in the control unit, to apply the generated alternating current voltage signal to the touch sensor via the insulated section of the touch sensor apparatus and to detect an influence on the applied alternating current voltage signal caused by the operator control event. An artificial alternating current voltage signal, the voltage and frequency of which is known for example, is therefore applied, wherein said signal is not applied for example directly at the control unit or the touch sensor but is transmitted via the insulated section and the control unit to the touch sensor. For this purpose the alternating current voltage signal can be applied for example to a ground connection of the control unit. This causes the wanted signal describing the operator control event and transmitted by the touch sensor to the control unit on the one hand and the applied alternating current voltage signal on the other hand to interact with one another, as a result of which the alternating current voltage signal induces a modulation of the wanted signal of the touch sensor.

The fault detection facility is designed to check whether the operator control event is detectable via the fault detection facility but not via the control unit, and if this is the case, i.e. if the operator control event can be detected by the fault detection facility but not via the control unit, the defect in the control unit is detected. A means is therefore provided by the fault detection facility to determine whether the operator control event has been detected at all at the user interface via the control unit or not. If the control unit does not detect an operator control event even though one has taken place, which is indicated on the basis of the influence of the alternating current voltage signal, it is assumed that the control unit has a defect. The defect of the control unit may alternatively be referred to as a fault and/or failure of the control unit. The defect of the control unit constitutes a fault relating to the control unit.

If the defect is identified, an alert message and/or warning message can for example be output to the user and/or a deactivation of the medical device or at least of a predefined function of the medical device can be triggered.

Via the fault detection facility, a checking entity is therefore provided which checks the function of the control unit and identifies whether a malfunction caused by other apparatuses is present in the form of the common mode interference and/or whether the control unit itself is defective and consequently limited in terms of its function. In this case the common mode interference is considered as a fault affecting the control unit since it leads to a distortion of the detected operator control event, whereas the defect in the control unit leads to the operator control event generally, at least temporarily, no longer being detectable via the control unit. A fault relating to the control unit of the touch sensor apparatus can therefore be reliably detected means of the touch sensor apparatus.

An exemplary embodiment relating to feature (a) provides that the fault detection facility comprises at least one capacitor, which is arranged between a voltage drop unit of the fault detection facility and the insulated power supply unit. The fault detection facility also comprises the voltage drop unit. The voltage drop unit may alternatively be referred to as a measurement impedance or measurement impedance unit. The voltage drop unit is a component at which the voltage between the first and second reference potential drops and consequently dips. The voltage drop unit may alternatively be referred to as a voltage dip unit. The voltage drop unit can comprise at least one resistor, at least one coil and/or at least one capacitor and is designed to determine the voltage dropping or dipping at the voltage drop unit via a comparison between the voltage upstream of the voltage drop unit and the voltage downstream of the voltage drop unit.

In a preferred example, the capacitor is a Y capacitor or a different type of capacitor which is designed for example to be connected in parallel with at least one transformer and/or a different insulating element of the touch sensor apparatus, wherein in particular at least one predefined safety requirement for the touch sensor apparatus is met.

The fault detection facility comprises a voltage detection unit which is designed to detect and quantify the voltage dipping or dropping at the voltage drop unit, that is to say the voltage between the first reference potential and the second reference potential. In addition, the fault detection facility comprises an evaluation unit which is designed to evaluate the voltage detected via the voltage detection unit. The voltage detection unit and the evaluation unit may be two components separate from one another or may be understood as a common voltage detection and evaluation unit. Alternatively, the voltage detection unit may be understood as a measurement apparatus for measuring the voltage and the evaluation unit as an analysis apparatus for evaluating the detected voltage. In one example, the evaluation unit can have an electrical connection to the control unit and via this transmit for example data, such as, for example, an actuation signal, to the control unit of the touch sensor apparatus. The fault detection facility therefore comprises at least the at least one capacitor, the voltage drop unit, such as, for example, the measurement impedance, the voltage detection unit and the evaluation unit, in order to enable the above-described functionality to be provided, that is to say the detection of the voltage that describes the common mode interference.

It can be provided that the capacitor is for example electrically coupled, in particular connected, to the voltage drop unit on a first side and to a connection to the ground of the insulated power supply unit on an opposite second side. Further, the voltage drop unit can be connected on a side facing away from the capacitor to a connection to the ground of the control unit, such as, for example, an earth connection or ground connection that is coupled to the control unit. By this means it becomes clear how the fault detection facility may be designed in detail for detecting the common mode interference.

Another exemplary embodiment comprises that the evaluation unit is designed to check, when evaluating the detected voltage, whether the latter exceeds a predefined voltage limit value. The evaluation unit is further designed to provide a trigger signal for at least one of the following measures if the detected voltage lies above the predefined voltage limit value. The trigger signal may alternatively be referred to as an actuating command for the control unit and is transmitted for example by the evaluation unit to the control unit. A further prerequisite for providing the measure may be that the touch sensor and the control unit are designed to perform a capacitive detection of the operator control event.

One measure may comprise that a change in a frequency used by the touch sensor for detecting the operator control event is made such that the frequency then used by the touch sensor is different from a frequency assigned to the detected voltage which exceeds the predefined voltage limit value, i.e. which is assigned to the detected common mode interference. The measure therefore comprises that, for example, an alternating current voltage frequency that is applied at the user interface so that the operator control event at the user interface is detected capacitively via the touch sensor is changed so that this cannot be confused with the frequency of the common mode interference. It can be provided that the frequency to which the change is made and the frequency of the common mode interference must be different by a predefined minimum frequency difference.

Alternatively or in addition, the measure may comprise ignoring the detected operator control event as long as the detected voltage lies above the predefined voltage limit value. It can therefore be specified to the control unit for example that currently, due to the sufficiently large common mode interference, any operator control event detected is not taken further into account. The event can therefore be deleted or overwritten, for example. Alternatively thereto, ignoring the event may comprise that currently no operator control event is detected. The event is ignored temporarily while the detected voltage lies above the predefined voltage limit value. The ignoring can end as soon as the detected voltage falls below the predefined voltage limit value or corresponds to the predefined voltage limit value. The result of this is that temporarily no further operator control event of a user can be detected in order to prevent nonexistent operator control events that are simulated only due to the common mode interference from having an impact on, for example, the medical device.

Alternatively or in addition thereto, one measure may provide that a distance mode is deactivated or paused. In the distance mode, an operator control event carried out at a distance from the user interface can be detected. The distance mode can be an add-on function of the touch sensor and for example allow remote operator control of the touch sensor apparatus. If such a distance mode is present, this can be switched off or stopped since it typically requires a low signal-to-noise ratio which cannot be achieved at the voltage above the predefined voltage limit value.

Alternatively or in addition thereto, the measure may comprise a deactivation or pausing of a glove mode in which an operator control event carried out by a user wearing a glove can be detected. The usability with gloves for example requires a low signal-to-noise ratio which cannot be achieved at a high level of common mode interference, i.e. at the voltage above the predefined voltage limit value, for which reason the glove mode is at least temporarily switched off or stopped. As soon as the detected voltage falls below the predefined voltage limit value or corresponds to the predefined voltage limit value, the distance mode and/or the glove mode can be reactivated.

Alternatively or in addition, it may be provided as the measure that an alert message is output. The alert message can be displayed on the user interface, for example. Alternatively or in addition thereto, the alert message can be output acoustically and/or haptically, for example via a vibration of the user interface. The alert message draws attention to the fault caused by the common mode interference, i.e. it indicates that for example another apparatus in the environment affects and possibly distorts the detection of the operator control event via the touch sensor apparatus. Furthermore, the alert message may request a deactivation or pausing of the other apparatus in order at least to reduce the common mode interference. The alert message may further comprise for example that currently the glove mode and/or the distance mode are/is deactivated or paused and/or that currently an ignoring of operator control events takes place on account for example of the common mode interference.

Alternatively or in addition, it may be provided as a measure that at least one interference-reducing method is activated, in particular a frequency spreading technique. It is therefore possible to have recourse to spread spectrum technology in order to counteract and for example at least to some extent suppress the common mode interference. However, disadvantages of such interference-reducing methods may be that the operability and consequently the touch sensor are affected, a power consumption is increased and/or at least one further disadvantage occurs. It can therefore be provided for example that this measure is adopted only if the detected voltage exceeds a further voltage limit value that is greater than the voltage limit value.

Voltage limit values of different magnitudes can be provided for the individual measures. For example, a higher voltage limit value can be provided in order, for example, to trigger the activation of the interference-reducing method and/or the deactivation or pausing of the distance mode and/or the glove mode, compared for example with a voltage limit value as of which the change of frequency and/or the output of the alert message is triggered. Alternative combinations and/or other means of influencing the voltage limit values are possible. The voltage limit value is typically a value which is dependent on the control unit used and/or the touch sensor used. The same voltage limit value therefore cannot be provided for all touch sensor apparatuses in one example, but instead a voltage limit value is provided which may be high or low to different extents depending on touch sensor apparatus and/or other components of the touch sensor apparatus.

Another exemplary embodiment provides that the touch sensor apparatus comprises an intermediate component which is arranged between the control unit and the insulated power supply unit. The fault detection facility is designed to detect as the common mode interference the voltage that dips between the first reference potential referenced to the ground of the control unit and a third reference potential referenced to a ground of the intermediate component. The third reference potential can be coupled less strongly to a ground potential than the second reference potential, which is referenced to the insulated power supply unit. For example, the third reference potential lies between the first reference potential and the second reference potential. If the touch sensor apparatus comprises further components, a kind of intermediate potential, referred to here as the third reference potential, can therefore be provided so that for example the capacitor does not have to be coupled directly to the insulated power supply unit on one side but for example is initially coupled to the ground connection of the intermediate component and via this to the insulated power supply unit. A requirement, however, is that a potential difference is present between the first ground and the third ground and that the third ground is insulated from the control unit and the touch sensor and is therefore assigned to the insulated section. This permits a versatile arrangement of the fault detection facility in the touch sensor apparatus.

An exemplary embodiment for feature (a) further comprises that the first reference potential is designed referenced to a local ground of the control unit which is provided via a ground connection, i.e. a ground connection or earth connection, between an energy supply unit of the touch sensor apparatus and the control unit. For example, the energy supply unit can be designed to convert the alternating current voltage provided by the insulated power supply unit into a direct current voltage and to provide the direct current voltage via a voltage supply connection between the energy supply unit and the control unit.

In one example, the voltage drop unit of the fault detection facility is now coupled to the ground connection, i.e. taps the latter, wherein as a result of the connection to the control unit and the connection of the control unit to the touch sensor it is to be expected that the common mode interference and not the wanted signal affects the ground connection between the energy supply unit and the control unit and leads there for example to an increase in potential. This illustrates an easy-to-implement way to provide the connection to the ground of the control unit.

In connection with feature (b), one embodiment provides that the fault detection facility comprises a further control unit. The further control unit is designed to generate the alternating current voltage signal. The further control unit can be designed for example as a capacitive control unit for a capacitive detection of the operator control event. The further control unit generates the alternating current voltage signal and forwards it to the insulated section of the touch sensor apparatus, i.e. it transmits the signal to the insulated section. The alternating current voltage signal is specified referenced to a ground of the further control unit. The further control device comprises for example a connection to ground, in particular a connection to the ground potential or to the intermediate potential. The alternating current voltage signal is therefore not influenced for example by the common mode interference caused by the other apparatus, if this is present, and leads to no influencing of the control unit and the touch sensor such that a beneficial way of generating the alternating current voltage signal is provided.

In another exemplary embodiment, the further control unit is designed for a capacitive detection of the operator control event, whereas the touch sensor and the control unit are designed for a resistive detection of the operator control event. According to feature (b), therefore, the functioning of the resistive control unit can be checked. This is advantageous since such control units can be particularly susceptible to defects. The further control unit itself has no separate touch sensor of its own but, like the control unit, uses the touch sensor of the touch sensor apparatus.

Alternatively or in addition thereto, the touch sensor and the control unit may likewise be designed for a capacitive detection of the operator control event, in which case an insulation is then assumed between the further control unit and the control unit. Versatile combinations of further control unit and control unit are therefore possible.

It can further be provided in an exemplary embodiment that at least one capacitor is arranged between the further control unit and the insulated section of the touch sensor apparatus. The capacitor is a Y capacitor, for example. The at least one capacitor can have a capacitance between 1 and 10 nanofarads, for example. The fault detection facility is designed to measure the effect of the applied alternating current voltage signal due to the operator control event by a current increase in at least one capacitor. This illustrates how the influence of the applied alternating current voltage signal caused by the operator control event is detected via the fault detection facility. In this arrangement the capacitor is an inexpensively available as well as space-saving component.

In another exemplary embodiment, the fault detection facility is designed to determine whether the detected current increase detected at the capacitor is greater than a predefined current increase limit value. If, in particular only if, the detected current increase is greater than the predefined current increase limit value, is the operator control event registered as such. The fault detection facility therefore detects the operator control event as an operator control event only when the detected current increase exceeds the predefined current increase limit value. The current increase limit value can in turn be dependent for example on the control unit or the touch sensor and consequently on the touch sensor apparatus. As soon as the current increase is greater than the current increase limit value, the defect in the control unit is to be considered as identified. The identification of the defect in the control unit with the aid of feature (b) is therefore based on relatively easy-to-implement measurements within the fault detection facility and is therefore particularly advantageous.

An exemplary embodiment may further comprise that, with regard to feature (b), the fault detection facility is designed to spread the alternating current voltage signal via frequency spreading across a frequency range with a predefined minimum frequency range width. This is beneficial primarily when the control unit and the touch sensor are provided for example as a multi-touch system, that is to say as a touch sensor which is able to detect multiple touches on the user interface as operator control events simultaneously. A multi-touch control device and a multi-touch touch sensor often use not just one predefined frequency but a frequency range comprising multiple individual frequencies. The multi-touch system is therefore relatively sensitive to interference signals such that the alternating current voltage signal is distributed, and therefore spread, over a plurality of frequencies.

Another exemplary embodiment provides that the touch sensor apparatus comprises a first touch sensor having a first control unit, the first touch sensor and the first control unit being designed for a capacitive determination of the operator control event. Furthermore, the touch sensor apparatus may comprise a second touch sensor having a second control unit, the second touch sensor and the second control unit being designed to detect an input of force in order to determine the operator control event. It is therefore provided that a capacitive touch sensor and a force sensor are provided. The second touch sensor and the second control unit are therefore designed to determine the operator control event on the basis of an input of force as an operator control event. Alternatively or in addition thereto, the second touch sensor and the second control unit can be designed for resistive detection of the operator control event.

The fault detection facility is designed to identify at least one defect in the second control unit, i.e. in the control unit for the force sensor. It can therefore be provided that two ways of detecting the operator control event are combined in the same touch sensor apparatus such that, for example, the operator control event can be detected via one of the combinations of touch sensor and control unit in each case both when the user interface is pressed and when the user interface is touched. Via the fault detection facility it is now possible for example to monitor only one of said two combinations of touch sensor and control unit with regard to a defect in the control unit in order, for example, to provide an increased safety requirement for certain functions of the medical device, but not to monitor every operator control event, such that, for example, the capacitive control unit for capacitive detection of the operator control event is not monitored according to feature (b) with regard to the defect in the capacitive control unit. This leads to a reasonably priced touch sensor apparatus that can be realized with lower investment of resources since a focus can be laid on just one part of the touch sensor apparatus.

An exemplary embodiment further provides that the user interface is subdivided into at least one first subsection and at least one second subsection. The touch sensor apparatus is designed to detect the operator control event in the at least one first subsection, in particular only, via the first touch sensor and the first control unit and to detect the operator control event in at least one second subsection, in particular only, via the second touch sensor and the second control unit. The user interface can for example comprise a first half and a second half, wherein on the first half for example only operator control gestures which comprise a touching of the user interface and so are capacitively detectable are taken into consideration, whereas in the second half and therefore in the second subsection only force inputs and therefore a pressing on the user interface are detectable as an operator control event. Alternatively to the first and second half, first and second subsections of any shape can be provided which for example can be arranged on the user interface alternately and/or reciprocally at least partially surrounding each other. In a preferred example, precisely one first subsection and precisely one second subsection can be provided. This leads to a clear and intuitively understandable spatial separation between the first and the second subsection.

One reason for this exemplary embodiment is at least that force sensors, though typically having a lower spatial resolution than for example capacitive touch sensors, are more resistant to damp fingers or liquids, for example. The second subsection can therefore be operated for example with moist or wet fingers and/or when liquid is present on the user interface, in contrast to the first subsection. A particularly robust embodiment of the touch sensor apparatus against wetting with a liquid or moisture and/or operator control events in combination with liquids or dampness is therefore achieved. In addition to the identification of the defect in the control unit, there is therefore the possibility that no erroneous operator control action is triggered due to liquids on the user interface since the force sensor is able to detect the operator control event regardless of liquids.

In an additional exemplary embodiment, the at least one second subsection is provided for operator control events relating to at least one safety-relevant function of a medical device for which the operator control event can be determined via the touch sensor apparatus. If, for example, the medical device is an X-ray device, an activation of X-ray radiation for example can only ever be triggered when the second subsection is operated. An emergency stop and/or emergency shutdown button, for example, may also be understood as a safety-relevant function and should be operable in the second subsection.

One or more example embodiments relates to a medical device comprising the touch sensor apparatus, as is described above.

One or more example embodiments relates to a method for operator control of a touch sensor apparatus for a medical device. The touch sensor apparatus corresponds to the above-described touch sensor apparatus, i.e. the latter comprises a touch sensor, a control unit via which an operator control event at a user interface of the touch sensor apparatus is detected by evaluation of sensor data of the touch sensor, and a fault detection facility for detecting a fault relating to the control unit. The fault detection facility directly measures a common mode interference if the fault is caused by common mode interference. This entails detecting a voltage dipping between a first reference potential referenced to a ground of the control unit and a second reference potential referenced to a ground of an insulated power supply unit of the touch sensor apparatus. Alternatively or in addition thereto, if the fault is caused by the defect in the control unit, the fault detection facility can generate an alternating current voltage signal, apply the generated alternating current voltage signal to the control unit via an insulated section of the touch sensor apparatus, detect an influence of the applied alternating current voltage signal caused by the operator control event, and check whether the operator control event is detectable via the fault detection facility but not via the control unit, in which event, if this is the case, the defect is identified in the control unit.

The touch sensor apparatus according to one or more example embodiments can be provided for a medical device or be incorporated in the latter. Alternatively thereto, the touch sensor apparatus can be incorporated in any other device or any other apparatus or be provided for these. Applications in other technical fields are therefore possible, such as, for example, in a household appliance, a mobile terminal device, a vehicle, and/or in a consumer electronics device.

The exemplary embodiments described in connection with the touch sensor apparatus apply analogously, where applicable, to the medical device according to one or more example embodiments and the method according to one or more example embodiments.

In the present disclosure, the control unit and/or the evaluation unit can be designed in each case as a data processing system or as a part of a data processing device. By a data processing device may be understood in particular a data processing device that contains a processing circuit. The data processing device can therefore process in particular data for performing computational operations. These may also include operations for performing indexed accesses to a data structure, for example a look-up table (LUT), in the same way as a data processing process implemented in hardware.

The data processing device may in particular contain one or more computers, one or more microcontrollers and/or one or more integrated circuits, for example one or more application-specific integrated circuits (ASICs), one or more field-programmable gate arrays (FPGAs), and/or one or more systems on a chip (SoCs). The data processing device may also contain one or more processors, for example one or more microprocessors, one or more central processing units (CPUs), one or more graphics processing units (GPUs) and/or one or more signal processors, in particular one or more digital signal processors (DSPs). The data processing device may also include a physical or virtual constellation of computers or other of the cited units.

In different exemplary embodiments, the data processing device contains one or more hardware and/or software interfaces and/or one or more memory units.

A memory unit may be embodied as a volatile data memory, for example as a dynamic random access memory (DRAM) or a static random access memory (SRAM), or as a non-volatile data memory, for example as a read-only memory (ROM), as a programmable read-only memory (PROM), as an erasable programmable read-only memory (EPROM), as an electrically erasable programmable read-only memory (EEPROM), as a flash memory or flash EEPROM, as a ferroelectric random access memory (FRAM), as a magnetoresistive random access memory (MRAM) or as a phase-change random access memory (PCRAM).

Independent of the grammatical term usage, individuals with male, female or other gender identities are included within the term.

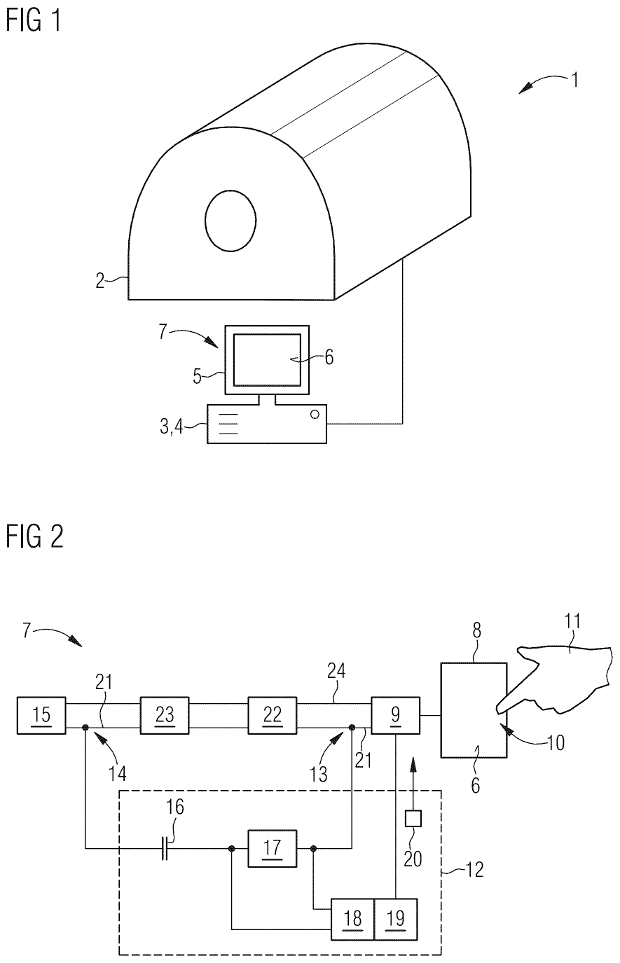

FIG. 1 schematically shows an exemplary embodiment of a medical device 1 which is designed, purely by way of example, as a magnetic resonance tomography (MRT) unit. The illustrated medical device 1 therefore comprises an MRI scanner 2 and a data processing system 3 for controlling the MRI scanner 2. The medical device 1 further comprises a computing unit 4 which is coupled to the data processing system 3 and/or the MRI scanner 2. Alternatively, the computing unit 4 may also comprise the data processing system 3 or vice versa. Other embodiments of the medical device 1 are possible, for example in the form of a computed tomography (CT) unit, etc.

To allow operator control of the medical device 1, a screen 5 may be provided which is designed for example as a touch sensor apparatus 7 or which may comprise such an apparatus. A user interface 6 of the screen 5 may therefore be provided such that touching and/or pressing the user interface 6 and/or an effect of force acting on the user interface 6 is detected as an operator control event 10 (see reference sign 10 in FIG. 2) of a user of the medical device 1.

FIG. 2 shows components of the touch sensor apparatus 7. The touch sensor apparatus 7 comprises a touch sensor 8 for example. In addition, it comprises a control unit 9 which is designed to detect the operator control event 10, which is initiated for example via a finger of a hand 11 of the user, at or on the user interface 6 of the touch sensor apparatus 7 by evaluating sensor data of the touch sensor 8. The touch sensor apparatus 7 further comprises a fault detection facility 12 which is provided for detecting a fault relating to the control unit 9. The control unit 9 and/or the fault detection facility 12 are/is for example incorporated in the data processing system 3 and/or the computing unit 4 or provided in addition to these.

FIG. 2 illustrates a feature (a) of the touch sensor apparatus 7 in detail, which feature concerns the case in which the fault relating to the control unit 9 is caused by common mode interference. According to feature (a), the fault detection facility 12 is designed to measure the common mode interference directly by detecting a voltage dipping between a first reference potential referenced to a ground 13 of the control unit 9 and a second reference potential referenced to a ground 14 of an insulated power supply unit 15 of the touch sensor apparatus 7.

The fault detection facility 12 can comprise at least one capacitor 16 as well as a voltage drop unit 17. The capacitor 16 is arranged between the voltage drop unit 17, which may be understood for example as a measurement impedance, and the insulated power supply unit 15. The capacitor 16 can be arranged for example with a ground connection 21, i.e. connected to earth, between an insulating element 23, which insulates the touch sensor apparatus 7 from the power supply unit 15, and the power supply unit 15 electrically insulated by the insulating element 23 from the rest of the touch sensor apparatus 7.

In addition, the fault detection facility 12 may comprise a voltage detection unit 18 which is designed to detect the voltage dipping at the voltage drop unit 17. The fault detection facility 12 may further comprise an evaluation unit 19 which is designed to evaluate the voltage detected via the voltage detection unit 18. The evaluation unit 19 is designed for example to check, when evaluating the detected voltage, whether this exceeds a predefined voltage limit value. If the voltage lies above the predefined voltage limit value, a trigger signal 20 can be provided for at least one of the following measures:
a change in a frequency that the touch sensor 8 uses to determine the operator control event 10 such that this used frequency differs from a frequency assigned to the detected voltage which lies above the predefined voltage limit value;
an ignoring of the identified operator control event 10 as long as the detected voltage lies above the predefined voltage limit value;
a deactivation and/or pausing of a distance mode in which an operator control event 10 carried out at a distance from the user interface 6 can be detected;
a deactivation and/or pausing of a glove mode in which an operator control event 10 carried out by a user wearing a glove can be detected;
an outputting of an alert message, for example on the user interface 6, that draws attention to the fault caused by the common mode interference; and/or
an activation of at least one interference-reducing method, in particular a frequency spreading technique.

At least individual cited measures or a plurality of the cited measures can require that the touch sensor 8 and the control unit 9 are designed for a capacitive determination of the operator control event 10 and not, for example, for a resistive determination and/or a determination of the operator control event 10 by evaluation of an input of force by the user.

The first reference potential may be understood as referenced to a local ground 13 of the control unit 9. This is provided via a ground connection 21 between an energy supply unit 22 of the touch sensor apparatus 7 and the control unit 9, as is also depicted here. It is further indicated here by the reference sign 20 that the trigger signal 20 can be transmitted by the evaluation unit 19 to the control unit 9 so that the latter can perform the measure.

Also shown is a supply line 24 via which for example the energy supply unit 22 can supply the control unit 9 with electrical power.

The touch sensor apparatus 7 can include an intermediate component which can be arranged between the control unit 9 and the insulated power supply unit 15. The fault detection facility 12 can then for example detect the voltage dipping between the first reference potential and a third reference potential referenced to a ground of the intermediate component. The third reference potential is typically less strongly coupled to the ground potential than the second reference potential referenced to the ground 14 of the insulated power supply unit 15.

It can be provided that feature (a) is provided only when the touch sensor 8 and the control unit 9 are designed for the capacitive determination of the operator control event 10. Alternatively or in addition thereto, feature (a) may also be provided when the touch sensor 8 and the control unit 9 are designed for the resistive determination and/or the determination of the operator control event 10 by evaluation of an input of force by the user.

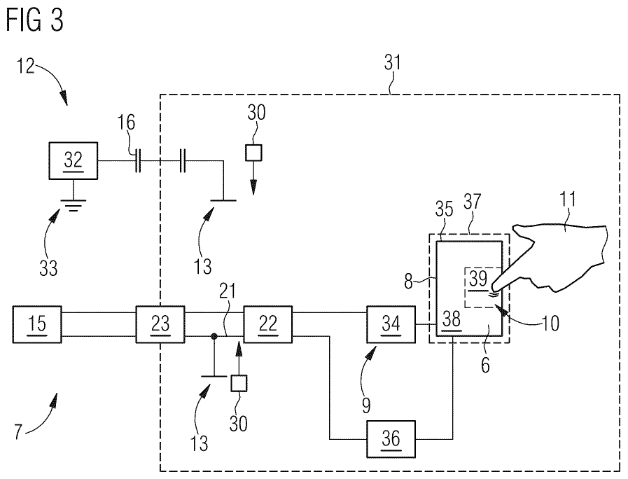

FIG. 3 shows a feature (b) of the touch sensor apparatus 7 in detail. The feature (b) is provided for the case in which the fault relating to the control unit 9 is caused by a defect in the control unit 9. According to feature (b), the fault detection facility 12 is designed to generate an alternating current voltage signal 30, to apply the generated alternating current voltage signal 30 to the touch sensor 8 via an insulated section 31 of the touch sensor apparatus 7, to detect an influence of the applied alternating current voltage signal 30 caused by the operator control event 10, and to check whether the operator control event 10 is detectable via the fault detection facility 12 but not via the control unit 9. If, for example, only the fault detection facility 12 detects the operator control event 10, but not the control unit 9, the defect in the control unit 9 is identified and consequently is present.

In connection with feature (b), the fault detection facility 12 can comprise a further control unit 32 which is designed to generate the alternating current voltage signal 30. The alternating current voltage signal 30 is for example referenced to a ground 33 of the further control unit 32, in particular the ground potential. It is further illustrated here by way of the depicted ground 13 of the control unit 9 how the alternating current voltage signal 30 can reach as far as the ground connection 21 between the insulating element 23 and the energy supply unit 22 via the insulated section 31, for example. Alternatively thereto, the ground connection 21 drawn in FIG. 2 can be provided between the energy supply unit 22 and the control unit 9.

In a preferred example, the further control unit 32 is designed for a capacitive determination of the operator control event 10. In this example, however, the touch sensor 8 and the control unit 9 are designed for a resistive determination of the operator control event 10.

In one example, at least one capacitor 16 can be arranged between the further control unit 32 and the insulated section 31. The fault detection facility 12 can comprise the capacitor 16. The fault detection facility 12 is designed for example to detect the influence of the applied alternating current voltage signal 30 by the operator control event 10 on the basis of a current increase in the at least one capacitor 16. The fault detection facility 12 is designed in particular to determine whether the current increase detected at the capacitor 16 is greater than a predefined current increase limit value. The operator control event 10 is only considered detected if, in particular only if, the detected current increase is greater than the predefined current increase limit value. Furthermore, the fault detection facility 12 can be designed to spread the alternating current voltage signal 30 via frequency spreading over a frequency range with a predefined minimum frequency width, in particular when the control unit 9 and the touch sensor 8 are provided for multi-touch detection.

Also outlined in FIG. 3 is the special case in which the touch sensor apparatus 7, in addition to the further control unit 32, also has a first control unit 34 for a first touch sensor 35 and a second control unit 36 for a second touch sensor 37. The first touch sensor 35 with the first control unit 34 is designed for example for the capacitive detection of the operator control event 10, in particular for a multi-touch detection. The second touch sensor 37 with the second control unit 36 is designed for example to detect an input of force in order to detect the operator control event 10 or for the resistive detection of the operator control event 10. In this example, the fault detection facility 12 can be designed at least to identify a defect in the second control unit 36. In addition or alternatively thereto, an identification of a defect in the first control unit 34 can be provided. In a preferred example, however, only the defect in the second control unit 36 is identified and taken into account.

The user interface 6 may comprise at least one first subsection 38 and at least one second subsection 39 that is different from the first subsection 38 and consequently can be subdivided into the first subsection 38 and the second subsection 39. The touch sensor apparatus 7 can be designed to detect the operator control event 10 in the at least one first subsection 38 via the first touch sensor 35 and the first control unit 34, though in the at least one second subsection 39 to detect the operator control event 10 via the second touch sensor 37 and the second control unit 36. At least the second subsection 39 can be provided for operator control events 10 relating to at least one safety-relevant function of the medical device 1 for which the touch sensor apparatus 7 determines the operator control event 10.

In this case, purely by way of example, a locally limited second subsection 39 is illustrated in which the user must press against the user interface 6 in order to perform an operator control action, whereas the surrounding sections of the user interface 6, which in this example are assigned to the first subsection 38, can be operable via touching, for example by tapping, swiping and/or another type of touch action and/or touch gesture.

It can be provided that for example two fault detection facilities 12 are incorporated in the touch sensor apparatus 7, and in fact one according to feature (a) and another according to feature (b), or that a common fault detection facility 12 comprises both the components for feature (a) and the components for feature (b).

All in all, the examples reveal an improvement in the noise suppression of capacitive touchscreens, i.e. of touch sensor apparatuses 7 for capacitive detection of the operator control event 10.

Feature (a) is based on making use of the knowledge that it is possible to measure the common mode interferences, i.e. the common-mode noise, directly. Since the common mode interferences are a potential difference between the capacitive measurement circuit (in this case given for example as control unit 9 with the touch sensor 8) and the preceding wiring or the ground potential, a current can flow through an additional measurement path from the measurement circuit to the previous potential, the measurement of which current alone results in the common mode interferences. This measurement takes place here via the fault detection facility 12. The design thereof comprises the following extensions compared with a typical touch sensor apparatus 7:
by at least one Y capacitor or a different capacitor 16 which can be connected as permitted by safety standards in parallel with transformers;
by a measurement impedance connected on one side to the ground connection 21 of the capacitive circuit (i.e. the sensor unit 9) and on the other side to the capacitor 16;
by a measurement and analysis apparatus (in this case the voltage detection unit 18 and the evaluation unit 19) which detects the voltage dipping at the measurement impedance and determines, for example via a limit value analysis, which frequencies in the common mode interference component are strong enough to lead potentially to errors in the detected operator control event 10.

In the next step, the measurement and analysis apparatus transmits the results of the analysis to the controller, in other words to the control unit 9. The latter is required to initiate different actions, i.e. take different measures, based on the common mode interferences present:
place the frequencies for the capacitive identification onto unused frequencies;
in the case of spontaneously occurring interferences simultaneously with touch inputs, ignore the identification until the frequencies are changed;
depending on noise level, activate or deactivate interference-suppressing features such as spread spectrum technology, the activation of which may involve disadvantages in terms of operator controllability, power consumption and other aspects;
in the case of a high noise level, deactivate extended touch features such as the usability with gloves or at a distance, for example, which require a particularly good signal-to-noise ratio;
signal to the user that strong interferences are present, and that the user is required to analyze their environment critically for potential sources of interference, that is to say to output the alert message.

Feature (b) is based on the fact that the capacitive wiring is not built separately on the electrically insulated part but over the insulation path, i.e. via the insulated section 31. In capacitive touch electronics, quite similarly to the regular layout, an alternating current voltage is generated, and consequently the alternating current voltage signal 30, in the further control unit 32. In the novel design, however, this is referenced to the ground potential and is applied via Y safety capacitors in the range of 1 to 10 nanofarads over the insulation path onto the ground potential of the resistive touch electronics. As a result, the entire insulated circuit is permanently modulated with this potential. When the resistive touch sensor is touched by a user, the capacitance of the sensor, and hence of the ground potential, is increased relative to the ground. This can be measured in capacitive touch electronics based on the increase in the current of the alternating current voltage into the Y capacitors, i.e. into the capacitors 16. An evaluation is therefore conducted on the transmitted current of the alternating current voltage of the capacitive touch electronics. An exceeding of a certain current increase limit value can be interpreted as an approach of a user.

Compared to the previous electronics, the novel design provides a reduced complexity and an improved functionality as a result of the omission of the timing coordination of the capacitive measurement system with the resistive measurement system: according to feature (b), both can be permanently on. With feature (b), the signal-to-noise ratio is also better and less dependent on parasitic system effects than the previous solution.

In the case of a multi-touch-capable capacitive input method using force sensors and a further redundant capacitive measurement method, the multi-touch-capable input method can only be used for example for non-safety-relevant inputs, and may therefore malfunction if conductive liquids are present on the screen 5. Safety-relevant inputs are identified exclusively via the liquid-insensitive force sensor and confirmed as a redundant measure by the additional capacitive measurement method, which is likewise insensitive to liquids.

A capacitive multi-touch electronics arrangement is present on the electronically insulated side, in this case the first control unit 34, connected to a capacitive touch sensor which is coupled to a glass surface that can be touched by the user. Also located here is the electronics for the force sensor, in this case the second control unit 36, which may be embodied differently. It is relevant that these components are pressure-activated sensors which are mounted beneath the glass surface touchable by the user and are also capable, when touched, of delivering X-Y coordinates accurate to 1 to 2 centimeters as well as the applied force.

The ground-referenced capacitive touch electronics arrangement is located on the non-insulated apparatus side, i.e. the further control unit 32, which is unable to supply X-Y coordinates but only identifies whether a change in ground capacitance due to a user is present. In the application here, this capacitive touch electronics arrangement must be coordinated with the capacitive multi-touch electronics arrangement and may only use frequencies for its alternating current voltage measurement which are not used by the capacitive multi-touch electronics arrangement since otherwise interference effects would be possible. In a preferred example, the signal is spread via spread band technology over a wide frequency range such that it can function with any multi-touch electronics arrangement. Via the connection of the insulated part via Y capacitors, the ground-referenced capacitive touch electronics likewise uses the capacitive touch sensor as a sensor surface.

The graphical user interface, i.e. the user interface 6, can be divided in each case into at least one non-safety-relevant section and one safety-relevant section, which are referred to here as the two subsections 38, 39. These can be next to each other, though a nesting and division into a number of sections in each case is also possible.

Each of the three installed sensors detects the whole screen in each case. Touch signals of the capacitive multi-touch sensor are, however, evaluated only for the non-safety-relevant section. Signals of the force sensor and the ground-referenced capacitive sensors are called upon only for the safety-relevant section. Only a few graphical elements are arranged in the safety-relevant section since known force sensors provide a resolution of 1 to 2 centimeters only. For this reason, only a small area centrally in each element is defined as a target section, and the elements are placed far enough from one another and from the non-safety-relevant section in order to rule out an operating error due to tolerance effects.

In other words, one or more example embodiments relates to a touch sensor apparatus 7 for a medical device 1, comprising: a touch sensor 8, a control unit 9, which is designed to detect an operator control event 10 at a user interface 6 of the touch sensor apparatus 7 by evaluating sensor data of the touch sensor 8, and a fault detection facility 12 for detecting a fault relating to the control unit 9. The fault detection facility 12 is designed to:
(a) measure the common mode interference directly if the fault is caused by a common mode interference; and/or,
(b) generate an alternating current voltage signal 30 if the fault is caused by a defect in the control unit 9, apply said signal to the touch sensor 8, detect an influence caused by the operator control event 10, and check whether the operator control event 10 is detectable via the fault detection facility 12 but not via the control unit 9, in which event, if this is the case, the defect in the control unit 9 is identified.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,“ ”connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A touch sensor apparatus for a medical device, comprising:
a touch sensor;
a controller configured to detect an operator control event at a user interface of the touch sensor apparatus by evaluating sensor data of the touch sensor; and
a fault detector configured to detect a fault relating to the controller, the fault detector configured to at least one of,
(a) measure a common mode interference directly if the fault is caused by the common mode interference, the fault detector configured to measure the common mode interference by detecting a voltage dipping between a first reference potential referenced to a ground of the controller and a second reference potential referenced to a ground of an insulated power supply unit of the touch sensor apparatus; or
(b) generate an alternating current voltage signal if the fault is caused by a defect in the controller, apply the generated alternating current voltage signal to the touch sensor via an insulated section of the touch sensor apparatus, detect an influence on the applied alternating current voltage signal caused by the operator control event, and check whether the operator control event is detectable via the fault detector and not via the controller, wherein the defect in the controller is identified if the operator control event is detectable via the fault detector and not via the controller.

2. The touch sensor apparatus of claim 1, wherein the fault detector comprises at least one capacitor between a voltage drop unit of the fault detector and the insulated power supply unit, a voltage detection unit is configured to detect a voltage dipping at the voltage drop unit, and an evaluation unit is configured to evaluate the voltage detected via the voltage detection unit.

3. The touch sensor apparatus of claim 2, wherein the evaluation unit is configured to check whether the detected voltage exceeds a predefined voltage limit value and, if the detected voltage is above a predefined voltage limit value, to provide a trigger signal for at least one of the following:
a change in a frequency that the touch sensor uses to determine the operator control event such that the frequency used differs from a frequency assigned to the detected voltage which lies above the predefined voltage limit value,
an ignoring of the determined operator control event when the detected voltage lies above the predefined voltage limit value,
a deactivation or pausing of a distance mode in which an operator control event carried out at a distance from the user interface is detectable,
a deactivation or pausing of a glove mode in which an operator control event carried out by a user wearing a glove is detectable,
an outputting of an alert message that draws attention to the fault caused by the common mode interference, or an activation of at least one interference-reducing method.

4. The touch sensor apparatus of claim 2, further comprising:
at least one intermediate component between the controller and the insulated power supply unit, wherein the fault detector is configured to detect, as the common mode interference, the voltage that dips between the first reference potential and a third reference potential referenced to a ground of the intermediate component, wherein the third reference potential is coupled less strongly to a ground potential than the second reference potential.

5. The touch sensor apparatus of claim 1, wherein the first reference potential is referenced to a local ground of the controller which is provided via a ground connection between an energy supply unit of the touch sensor apparatus and the controller.

6. The touch sensor apparatus of claim 1, wherein the fault detector comprises a further controller configured to generate the alternating current voltage signal, wherein the alternating current voltage signal is referenced to a ground of the further controller.

7. The touch sensor apparatus of claim 6, wherein the further controller is configured to perform a capacitive determination of the operator control event, and the touch sensor and the controller are configured to perform a resistive determination of the operator control event.

8. The touch sensor apparatus of claim 6, wherein at least one capacitor of the fault detector is between the further controller and the insulated section of the touch sensor apparatus and the fault detector is configured to detect an influence on the applied alternating current voltage signal due to the operator control event by a current increase in the at least one capacitor.

9. The touch sensor apparatus of claim 8, wherein the fault detector is configured to determine whether the detected current increase is greater than a predefined current increase limit value and, if the detected current increase is greater than the predefined current increase limit value, to detect the operator control event.

10. The touch sensor apparatus of claim 1, wherein the fault detector is configured to spread the alternating current voltage signal via frequency spreading over a frequency range with a predefined minimum frequency range width.

11. The touch sensor apparatus of claim 1, further comprising:
a first touch sensor having a first controller, the first controller configured to perform a capacitive detection of the operator control event; and
a second touch sensor having a second controller, the second controller configured to at least one of perform a resistive detection of the operator control event or determine the operator control event based on an input of force, and the fault detector configured to identify at least one defect in the second controller.

12. The touch sensor apparatus of claim 11, wherein the user interface is subdivided into at least one first subsection and at least one second subsection and the touch sensor apparatus is configured to detect the operator control event in the at least one first subsection via the first touch sensor and the first controller and to detect the operator control event in the at least one second subsection via the second touch sensor and the second controller.

13. The touch sensor apparatus of claim 12, wherein the at least one second subsection is provided for operator control events relating to safety-relevant functions of a medical device for which the operator control event is determinable via the touch sensor apparatus.

14. A medical device comprising:
the touch sensor apparatus of claim 1.

15. The touch sensor apparatus of claim 3, further comprising:
at least one intermediate component between the controller and the insulated power supply unit, wherein the fault detector is configured to detect, as the common mode interference, the voltage that dips between the first reference potential and a third reference potential referenced to a ground of the intermediate component, wherein the third reference potential is coupled less strongly to a ground potential than the second reference potential.

16. The touch sensor apparatus of claim 15, wherein the first reference potential is referenced to a local ground of the controller which is provided via a ground connection between an energy supply unit of the touch sensor apparatus and the controller.

17. The touch sensor apparatus of claim 15, wherein the fault detector comprises a further controller configured to generate the alternating current voltage signal, wherein the alternating current voltage signal is referenced to a ground of the further controller.

18. The touch sensor apparatus of claim 9, wherein the fault detector is configured to spread the alternating current voltage signal via frequency spreading over a frequency range with a predefined minimum frequency range width.

19. The touch sensor apparatus of claim 2, further comprising:
a first touch sensor having a first controller, the first controller configured to perform a capacitive detection of the operator control event; and
a second touch sensor having a second controller, the second controller configured to at least one of perform a resistive detection of the operator control event or determine the operator control event based on an input of force, and the fault detector configured to identify at least one defect in the second controller.

20. The touch sensor apparatus of claim 19, wherein the user interface is subdivided into at least one first subsection and at least one second subsection and the touch sensor apparatus is configured to detect the operator control event in the at least one first subsection via the first touch sensor and the first controller and to detect the operator control event in the at least one second subsection via the second touch sensor and the second controller.
